(19) United States
(12) Patent Application Publication
(10) Pub. No.: US 2024/0189160 A1
(43) Pub. Date: Jun. 13, 2024

(54) WEARABLE ABSORBENT ARTICLE

(71) Applicant: Anna Rivers, Daphne, AL (US)

(72) Inventor: Anna Rivers, Daphne, AL (US)

(21) Appl. No.: 18/199,930

(22) Filed: May 20, 2023

Related U.S. Application Data
(60) Provisional application No. 63/430,784, filed on Dec. 7, 2022.

(51) Int. Cl. A61F 13/496; A61F 13/49; A61F 13/494; A61F 13/66

(52) U.S. Cl. CPC A61F 13/496; A61F 13/49011; A61F 13/4946; A61F 13/66; A61F 2013/15121

(57) ABSTRACT

A wearable absorbent article, including a main body to be worn around at least a portion of a pelvis of a user, the main body including a pelvic covering portion having a size and a shape to cover an entirety of at least one of a crotch, genitalia, and buttocks of a user, a plurality of leg receiving apertures disposed on at least a portion of the pelvic covering portion to receive legs of the user therethrough, and a waist receiving aperture disposed on at least a portion of the pelvic covering portion to receive a waist of the user therethrough, a fluid absorbing pad disposed on and within at least a portion of the pelvic covering portion to absorb at least one fluid received thereon, and a plurality of absorbent liners disposed on at least a portion of the pelvic covering portion to prevent any leakage of the at least one fluid beyond a boundary of the fluid absorbing pad toward the plurality of leg receiving apertures.

### CROSS-REFERENCE TO RELATED APPLICATIONS

This application claims priority under 35 USC § 120 from U.S. Provisional Application No. 63/430,784, entitled “Wearable Absorbent Article,” which was filed on Dec. 7, 2022, in the United States Patent and Trademark Office, the disclosure of which is incorporated herein in its entirety by reference.

### BACKGROUND

### 1. Field

The present general inventive concept relates generally to undergarments, and particularly, to a wearable absorbent article.

### 2. Description of the Related Art

Diapers are a type of undergarment worn to absorb urine, feces, as well as other bodily fluids to prevent soiling of outer clothing. For an adult user, certain medical conditions can cause difficulty in control of bowel movements and/or incontinence that may lead to leakage of bodily fluids.

Unfortunately, adult diapers often do not provide adequate coverage around a body of the adult user and are uncomfortable to wear for long periods of time due to lack of adequate padding.

Therefore, there is a need for a wearable absorbent article that absorbs discharged bodily fluids, provides more coverage around the body, and can be worn for extended periods of time.

### SUMMARY

The present general inventive concept provides a wearable absorbent article.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a wearable absorbent article, including a main body to be worn around at least a portion of a pelvis of a user, the main body including a pelvic covering portion having a size and a shape to cover an entirety of at least one of a crotch, genitalia, and buttocks of a user, a plurality of leg receiving apertures disposed on at least a portion of the pelvic covering portion to receive legs of the user therethrough, and a waist receiving aperture disposed on at least a portion of the pelvic covering portion to receive a waist of the user therethrough, a fluid absorbing pad disposed on and within at least a portion of the pelvic covering portion to absorb at least one fluid received thereon, and a plurality of absorbent liners disposed on at least a portion of the pelvic covering portion to prevent any leakage of the at least one fluid beyond a boundary of the fluid absorbing pad toward the plurality of leg receiving apertures.

The fluid absorbing pad may be disposed between layers of the pelvic covering portion to be hidden within the pelvic covering portion.

The wearable absorbent article may further include an elastic waistband circumferentially disposed around at least a portion of a perimeter of a top edge of the pelvic covering portion to prevent the pelvic covering portion from moving away from the waist of the user.

The wearable absorbent article may further include a plurality of adhesive pads disposed on at least a portion of the pelvic covering portion removably connect the pelvic covering portion to an external garment.

The wearable absorbent article may further include an odor preventing layer disposed on and within at least a portion of the fluid absorbing pad to absorb an odor from the at least one fluid therein.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a side perspective view of a wearable absorbent article, according to an exemplary embodiment of the present general inventive concept; and

FIG. 2 illustrates a zoomed in view of the wearable absorbent article, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Wearable Absorbent Article 100
Main Body 110
Pelvic Covering Portion 111
Leg Receiving Apertures 112
Waist Receiving Aperture 113
Fluid Absorbing Pad 120
Absorbent Liners 130
Elastic Waistband 140
Adhesive Tabs 150
Odor Preventing Layer 160
Fitted Lining 170

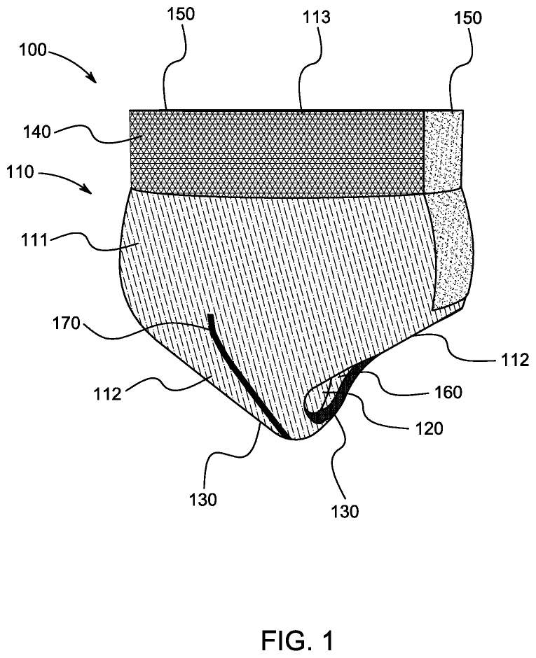

FIG. 1 illustrates a side perspective view of a wearable absorbent article 100, according to an exemplary embodiment of the present general inventive concept.

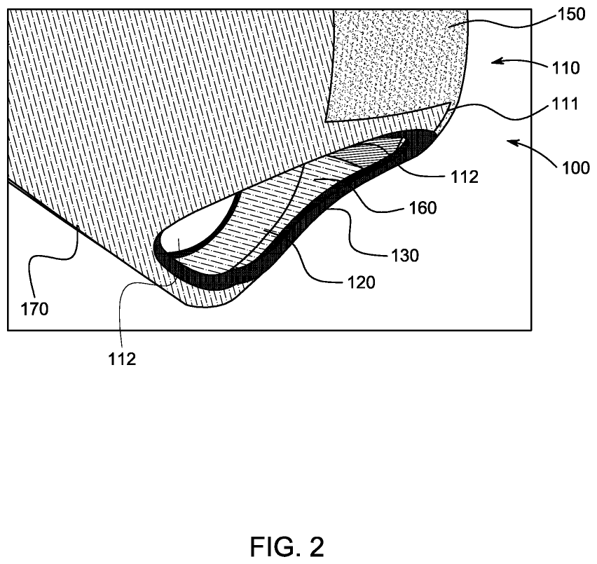

FIG. 2 illustrates a zoomed in view of the wearable absorbent article 100, according to an exemplary embodiment of the present general inventive concept.

The wearable absorbent article 100 may be constructed from at least one of cloth, plastic, wood, and rubber, etc., but is not limited thereto.

The wearable absorbent article 100 may include a main body 110, a fluid absorbing pad 120, a plurality of absorbent liners 130, an elastic waistband 140, a plurality of adhesive pads 150, an odor preventing layer 160, and a fitted lining 170, but is not limited thereto.

Referring to FIGS. 1 and 2, the main body 110 is illustrated to be briefs. However, the main body 110 may be boxer shorts, boxer briefs, panties, or any other type of undergarment known to one of ordinary skill in the art, but is not limited thereto.

The main body 110 may include a pelvic covering portion 111, a plurality of leg receiving apertures 112, and a waist receiving aperture 113, but is not limited thereto.

The pelvic covering portion 111 may be removably disposed around at least a portion of a body of a user. More specifically, the pelvic covering portion 111 may be worn around at least a portion of a pelvis of the user to cover the pelvis of the user. Additionally, the pelvic covering portion 111 may have a size and/or a shape to surround an entirety of a crotch, genitalia, and buttocks.

The plurality of leg receiving apertures 112 may be disposed on at least a portion of the pelvic covering portion 111. A first of the plurality of leg receiving apertures 112 may be disposed on a first side of the pelvic covering portion 111 and a second of the plurality of leg receiving apertures 112 may be disposed on a second side of the pelvic covering portion 111 opposite with respect to the first side. Each of the plurality of leg receiving apertures 112 may receive at least one leg of the user therethrough.

The waist receiving aperture 113 may be disposed on at least a portion of the pelvic covering portion 111. The waist receiving aperture 113 receive a waist of the user therethrough.

The fluid absorbing pad 120 may be constructed of cotton, hemp, bamboo, and/or any combination thereof, but is not limited thereto.

The fluid absorbing pad 120 may be disposed on and/or within at least a portion of the pelvic covering portion 111. The fluid absorbing pad 120 may be disposed on a fabric of the pelvic covering portion 111 and/or integrated within material of the pelvic covering portion 111, such that the fluid absorbing pad 120 may be disposed between layers of the pelvic covering portion 111 to be hidden within the pelvic covering portion 111. The fluid absorbing pad 120 may absorb at least one fluid (e.g., urine, feces, water, blood, oil, etc.) received thereon. Also, the fluid absorbing pad 120 may prevent the at least one fluid from spreading beyond a boundary of the fluid absorbing pad 120, such as the pelvic covering portion 111.

The plurality of absorbent liners 130 may be disposed on at least a portion of the pelvic covering portion 111. A first of the plurality of absorbent liners 130 may be disposed on the first side of the pelvic covering portion 111 and a second of the plurality of absorbent liners 130 may be disposed on the second side of the pelvic covering portion 111 opposite with respect to the first side. The plurality of absorbent liners 130 may prevent any leakage of the at least one fluid beyond the boundary of the fluid absorbing pad 120 toward the plurality of leg receiving apertures 112. Additionally, the plurality of absorbent liners 130 may cushion the at least one leg of the user within each of the plurality of leg receiving apertures 112.

The elastic waistband 140 may be circumferentially disposed around at least a portion of a perimeter of a top edge of the pelvic covering portion 111. The elastic waistband 140 may be flexible. In other words, the elastic waistband 140 may at least partially deform (e.g., bend, stretch, expand, contract) in response to an application of force (e.g., pushing, pulling, worn around the waist of the user) thereto. Also, the elastic waistband 140 may be biased to contract to a predetermined size (e.g., a size based on construction, such as small, medium, large, and/or any other size determined by a manufacturer). As such, the elastic waistband 140 may prevent the pelvic covering portion 111 from moving away (i.e., falling off) from the waist of the user during use.

Each of the plurality of adhesive pads 150 may include an adhesive surface (e.g., a tape, a glue) and an adhesive cover, but is not limited thereto.

The plurality of adhesive pads 150 may be disposed on at least a portion of the pelvic covering portion 111. A first of the plurality of adhesive pads 150 may be disposed on the first side of the pelvic covering portion 111 and a second of the plurality of adhesive pads 150 may be disposed on the second side of the pelvic covering portion 111 opposite with respect to the first side. The adhesive cover of each of the plurality of adhesive pads 150 may be removed (i.e., torn away) to expose the adhesive surface thereunder. As such, the adhesive pads 150 may removably connect to an external garment (e.g., pants, shorts, skirt, dress, etc.). Also, the adhesive pads 150 may prevent the adhesive pads 150 from moving away (i.e., falling off) from the external garment during use.

The odor preventing layer 160 may include oil, lotion, and/or any other combination of liquid, but is not limited thereto.

The odor preventing layer 160 may be disposed on and/or within at least a portion of the fluid absorbing pad 120. The odor preventing layer 160 may emit a fragrance therefrom and/or absorb an odor therein. In other words, the odor preventing layer 160 may prevent odor due to the at least one fluid absorbed by the fluid absorbing pad 120.

The fitted lining 170 may be disposed on at least a portion of the pelvic covering portion 111. The fitted lining 170 may be stitching to prevent separation between portions of the pelvic covering portion 111 where the stitching is placed, such that the stitching may cover the crotch and/or a line between buttocks of the user. As such, the fitted lining 170 may improve a fit of the pelvic covering portion 111 while worn by the user.

Therefore, the wearable absorbent article 100 may protect the user from leakage of bodily fluids. Also, the wearable absorbent article 100 may provide full coverage of the pelvis of the user while remaining comfortable for long periods of time.

The present general inventive concept may include a wearable absorbent article 100, including a main body 110 to be worn around at least a portion of a pelvis of a user, the main body 110 including a pelvic covering portion 111 having a size and a shape to cover an entirety of at least one of a crotch, genitalia, and buttocks of a user, a plurality of leg receiving apertures 112 disposed on at least a portion of the pelvic covering portion 111 to receive legs of the user therethrough, and a waist receiving aperture 113 disposed on at least a portion of the pelvic covering portion 111 to receive a waist of the user therethrough, a fluid absorbing pad 120 disposed on and within at least a portion of the pelvic covering portion 111 to absorb at least one fluid received thereon, and a plurality of absorbent liners 130 disposed on at least a portion of the pelvic covering portion 111 to prevent any leakage of the at least one fluid beyond a boundary of the fluid absorbing pad 120 toward the plurality of leg receiving apertures 112.

The fluid absorbing pad 120 may be disposed between layers of the pelvic covering portion 111 to be hidden within the pelvic covering portion 111.

The wearable absorbent article 100 may further include an elastic waistband 140 circumferentially disposed around at least a portion of a perimeter of a top edge of the pelvic covering portion 111 to prevent the pelvic covering portion 111 from moving away from the waist of the user.

The wearable absorbent article 100 may further include a plurality of adhesive pads 150 disposed on at least a portion of the pelvic covering portion 111 removably connect the pelvic covering portion 111 to an external garment.

The wearable absorbent article 100 may further include an odor preventing layer 160 disposed on and within at least a portion of the fluid absorbing pad 120 to absorb an odor from the at least one fluid therein.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A wearable absorbent article, comprising:
a main body to be worn around at least a portion of a pelvis of a user, the main body comprising:
a pelvic covering portion having a size and a shape to cover an entirety of at least one of a crotch, genitalia, and buttocks of a user,
a plurality of leg receiving apertures disposed on at least a portion of the pelvic covering portion to receive legs of the user therethrough, and
a waist receiving aperture disposed on at least a portion of the pelvic covering portion to receive a waist of the user therethrough;
a fluid absorbing pad disposed on and within at least a portion of the pelvic covering portion to absorb at least one fluid received thereon; and
a plurality of absorbent liners disposed on at least a portion of the pelvic covering portion to prevent any leakage of the at least one fluid beyond a boundary of the fluid absorbing pad toward the plurality of leg receiving apertures.

2. The wearable absorbent article of claim 1, wherein the fluid absorbing pad is disposed between layers of the pelvic covering portion to be hidden within the pelvic covering portion.

3. The wearable absorbent article of claim 1, further comprising:
an elastic waistband circumferentially disposed around at least a portion of a perimeter of a top edge of the pelvic covering portion to prevent the pelvic covering portion from moving away from the waist of the user.

4. The wearable absorbent article of claim 1, further comprising:
a plurality of adhesive pads disposed on at least a portion of the pelvic covering portion removably connect the pelvic covering portion to an external garment.

5. The wearable absorbent article of claim 1, further comprising:
an odor preventing layer disposed on and within at least a portion of the fluid absorbing pad to absorb an odor from the at least one fluid therein.
